(19) United States
(12) Patent Application Publication
(10) Pub. No.: US 2004/0015091 A1
(43) Pub. Date: Jan. 22, 2004

(54) System and method of assessment of arousal, pain and stress during anesthesia and sedation

(75) Inventors: Scott D. Greenwald, Norfolk, MA (US); Albert Dahan, Amsterdam (NL)

Correspondence Address: HALE AND DORR, LLP, 60 STATE STREET, BOSTON, MA, 02109

(73) Assignee: Aspect Medical Systems, Newton, MA

(21) Appl. No.: 10/404,869

(22) Filed: Apr. 1, 2003

Related U.S. Application Data
(60) Provisional application No. 60/369,142, filed on Apr. 1, 2002.

(51) Int. Cl. A61B 5/0402

(52) U.S. Cl. 600/513

(57) ABSTRACT

A PTT monitoring system for measuring arousal and responses to stress or pain during sedation or anesthesia. includes ECG electrodes and a PPG probe connected to a computer via signal conditioning and digitizing hardware. Lead I is typically used as the ECG lead while the PPG probe is typically placed on a finger. The ECG and PPG waveforms are continuously analyzed to update and display a current estimate of the subject's PPT from heart to hand. For each cardiac cycle, fiducial points are identified to indicate the pulse onset time (via QRS detection in the ECG) and pulse arrival time (via the point of steepest ascent in the PPG). The onset and arrival times for each cardiac cycle are paired, and the time difference is the interval estimate for that beat. An artifact post-processor (e.g., trim-mean filtering) excludes unlikely intervals from entering the averaged, current estimate of PTT. Finally, the current PTT estimate is displayed numerically and the trend of PTT is updated every second. Clinicians may interpret the instantaneous PTT value directly or in context of its recent trend. If there is a rapid decrease in PTT much less than the predetermined baseline value when the patient should be unconscious and free of stress and pain, then supplemental analgesics are administered to bring PTT greater than or equal to such baseline value.

### CROSS REFERENCE TO RELATED APPLICATION

This application claims priority from U.S. Provisional Application Serial No. 60/369,142 filed Apr. 1, 2002.

### FIELD OF THE INVENTION

The present invention relates to devices for analyzing autonomic tone in a body, and, more particularly, to devices for measuring arousal, stress and pain during sedation and anesthesia.

### BACKGROUND OF THE INVENTION

Management of anesthesia requires titration of medications to achieve adequate states of three clinical endpoints: consciousness (i.e. hypnotic state), analgesia, and muscle relaxation. Commercial devices currently exist to directly measure consciousness (e.g., Bispectral Index, Aspect Medical Systems, MA) and muscle relaxation. To date, clinicians indirectly monitor adequacy of analgesia (i.e., the lack of excessive stress or perceived pain) in unresponsive patients by assessing the autonomic state of their patient, traditionally via heart rate, blood pressure, sweating and/or tearing. During periods of arousal, stress or pain in normal subjects, there is a significant change in the autonomic state: there is an increase in sympathetic tone and a decrease in parasympathetic tone causing an increase in heart rate and arterial constriction (tone) resulting in increased blood pressure. During periods of relaxation, the opposite response typically occurs. Consequently, clinicians typically monitor heart rate and blood pressure as standard practice and note changes in these parameters in context with changes in interventions or stimulation.

This patent describes the novel application of the use of Pulse Wave Velocity (PWV) and Pulse Transit Time (PTT) to assess the autonomic state of the patient during anesthesia or sedation.

“Pulse Wave Velocity” (PWV) is the velocity of the wave front propagating along an arterial tree generated by a bolus of blood ejected from a ventricle. The PWV is inversely proportional to the tension in the arterial wall and moves more rapidly (4-5 m/sec) than the blood flow itself (<0.5 m/sec). “Pulse Transit Time” is the time for the wave front to travel a fixed distance (“D”), for example, from the root of the aorta to an index finger. The transit time is related to the velocity in the expected way: PTT=D/PWV.

One estimator of Pulse Transit Time is the time difference from initial ventricular contraction (as estimated by the peak of the R-wave within the electrocardiogram (ECG)) to the arrival of the resultant pulse at the periphery (as estimated by the point of steepest ascent of the photoplethysmography signal (PPG) measured at the finger (via a pulse oximetry device, for example.)) Although this estimator is biased (i.e., it is longer than necessary because it contains the period when the heart contracts prior to ejecting blood), this estimator is precise and readily calculated.

Because PTT and PWV are related to arterial tone, changes in these parameters reflect changes in the autonomic control of arterial tone. For example, during periods of increased sympathetic activity (e.g., in response to painful stimulation), arterial tone increases (i.e., arteries stiffen and compliance decreases). Consequently, PWV increases and PTT decreases. Conversely, during periods of decreased sympathetic activity or increased parasympathetic activity (e.g., as subjects fall unconscious), arterial tone decreases. Consequently, PWV decreases and PTT increases.

Because changes in PTT and PWV reflect changes in the autonomic system and in vascular stiffness (i.e., compliance), these parameters have been studied in various applications.

The principal object of the present invention is the use of the PTT to quantify the level of stress, pain and arousal of a subject.

Another object of the present invention to provide a method and device for accurately determining the PTT from the heart to the periphery.

### SUMMARY OF THE INVENTION

A PTT monitoring system is described for measuring arousal and responses to stress or pain during sedation or anesthesia. In a preferred embodiment, the PTT monitoring system includes ECG electrodes and a PPG probe connected to a computer via signal conditioning and digitizing hardware. Lead I is typically used as the ECG lead while the PPG probe is typically placed on a finger.

The ECG and PPG waveforms are continuously analyzed to update and display a current estimate of the subject's PPT from heart to hand. For each cardiac cycle, fiducial points are identified to indicate the pulse onset time (via QRS detection in the ECG) and pulse arrival time (via the point of steepest ascent in the PPG). The onset and arrival times for each cardiac cycle are paired, and the time difference is the interval estimate for that beat. An artifact post-processor (e.g., trim-mean filtering) excludes unlikely intervals from entering the averaged, current estimate of PTT. Finally, the current PTT estimate is displayed numerically and the trend of PTT is updated every second. Clinicians may interpret the instantaneous PTT value directly or in context of its recent trend. If there is a rapid decrease in PTT much less than the predetermined baseline value when the patient should be unconscious and free of stress and pain, then supplemental analgesics are administered to bring PTT greater than or equal to such baseline value.

These and other objects and features of the present invention will be more fully understood from the following detailed description which should be read in light of the accompanying drawings in which corresponding reference numerals refer to corresponding parts throughout the several views.

### BRIEF DESCRIPTION OF THE DRAWINGS

FIG. 1 is an illustration of a human body indicating the preferred ECG electrode and probe placements when using the data acquisition and analysis system of the present invention;

FIG. 2 is a schematic view of the ECG and PPG data acquisition and analysis system constructed according to the present invention;

FIG. 3 is a process flow diagram of the signal analysis method according to the present invention;

FIG. 4 is a schematic view of 3 seconds of ECG and PPG waveforms indicating the fiducial point locations within same.

FIG. 5 is a graph of a simultaneous trend of BIS and PPT over the course of a surgical case.

### DETAILED DESCRIPTION OF THE INVENTION

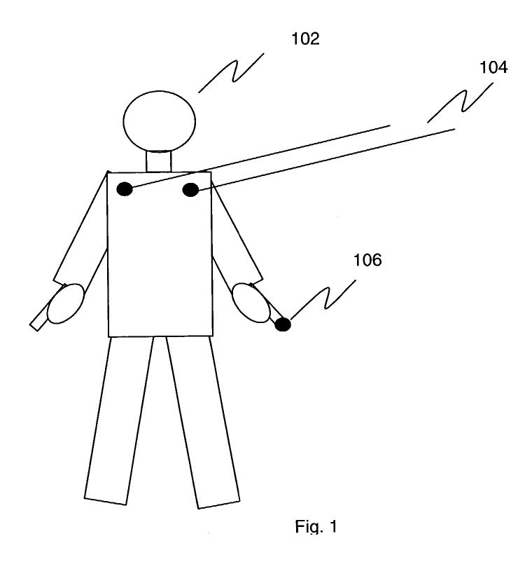

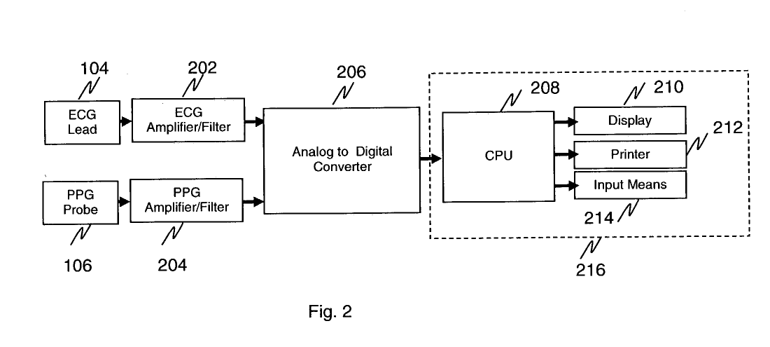

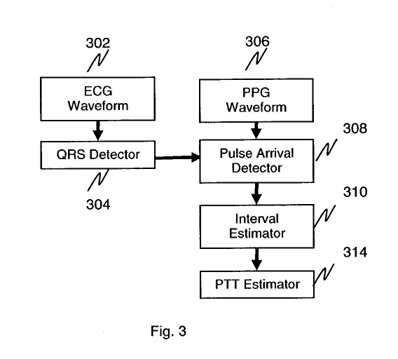

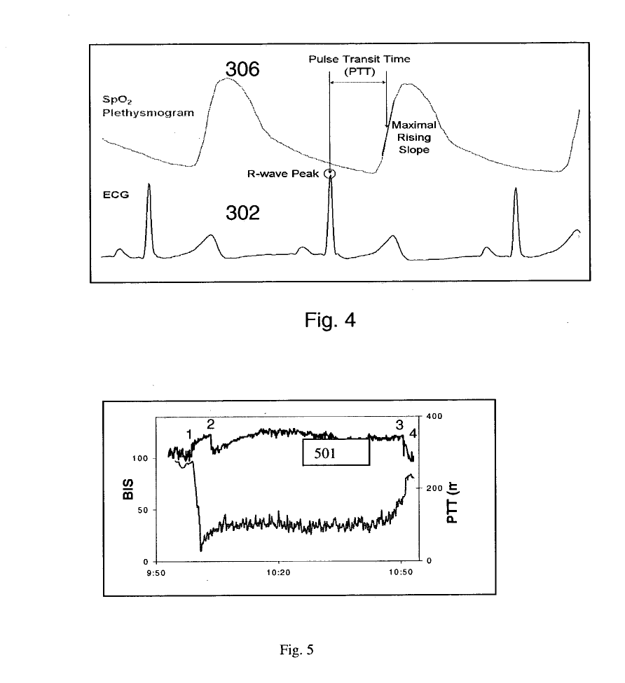

Referring to FIGS. 1 and 2, the PTT monitoring device 200 includes of a computer 216 (which includes CPU 208, display 210, printer 212, and input means 214) that analyzes digitized ECG and PPG waveforms extracted from a subject 102 via ECG leads 104 and PPG probe 106. The analog ECG and PPG signals collected from the body are first conditioned by the ECG amplifier/filter 202 and PPG amplifier/filter 204, respectively, prior to sampling by the analog-to-digital converter 206 for analysis by the CPU 208.

In the preferred embodiment, ECG lead 104 is Lead I measured across the patient's chest and the PPG probe 106 is an oximetry probe (e.g., Oxy-Tip+ by Datex-Ohmeda, Finland) placed on the subject's index finger. Pulse wave signals may also be acquired through a tonometer device or an invasive arterial line. In a preferred embodiment, the ECG signal conditioning amplifier/filter 202 is a 4-pole high pass filter with 3-db breakpoint at 0.05 Hz with gain adjusted so that 10 mv ECG is scaled to the full input range of the analog-to-digital converter 206. The PPG signal conditioning amplifier/filter is preferably a 4-pole high pass filter with 3-db breakpoint at 0.05 Hz and the gain is adjusted so that 100% SaO2 in the PPG waveform is scaled to the full input range of the analog-to-digital converter 206. For example, the ECG signal can be collected from the analog output pin #18 of a Datex-Ohmeda CardioCap II system. Likewise, the PPG signal can be collected from the analog output pin #22 of a Datex-Ohmeda Capnomax Ultima sytems.

Analog-to-digital conversion can be performed with any number of commonly available analog-to-digital converter cards installed in a computer or with the A1000 EEG Monitor (Aspect Medical Systems, Inc, Newton Mass.). The preferred sampling rate is 128 samples per second, and should be no less because of increased jitter in estimation of fidicual point placement.

For each cardiac cycle, the ECG waveform 302 and resulting PPG waveform 306 are analyzed to identify pulse onset and arrival times. QRS detector 304 determines the pulse onset time by detecting the peak of each R-wave using a matched filter with threshold as described below. The pulse arrival detector 308 determines the pulse arrival time by detecting the peak in the first derivative of each pulse response (i.e., the point of steepest ascent in the PPG waveform) using a matched filter with threshold as described below. For each detected R-wave, the interval estimator 310 determines the time interval for a given beat by measuring the difference in the pulse onset and arrival times. If no arrival time is detected within a maximal delay (typically 500 msec), then the interval is excluded from further analysis by the interval estimator 310. Finally, the PTT estimator 314 updates the current PTT estimate using the a trim-mean filter (using the central 50% of observations to exclude artifactual intervals) calculated over the preceding user-defined window (30 seconds in the preferred embodiment)

In the preferred embodiment, the peak detectors used for the QRS detector 304 and pulse arrival detector 308 employ matched filters with threshold, a common technique for peak detection. The method used in the preferred embodiment is described in: W. A. H. Engelse and C. Zeelenberg, “A single scan algorithm for QRS detection and feature extraction”, 1979 Computers in Cardiology 6:37-42 the teachings of which are incorporated herein. Software known as “sqrs.c” that implements this algorithm (for data sampled at 125 samples per second) is available from MIT researchers at http://www.physionet.org/physiotools/wfdb/app/sqrs.c. This method processes the input data stream from the analog-to-digital converter 206 continuously.

The computer display 210 is updated each second with the current numerical value as well as an update of the time course of the PTT (i.e., the PTT trend). Computer printer 212 is available to the user to record hardcopies of the PTT trend 501 shown in FIG. 5 for documenting a particular subject case.

An example of such a system for performing PTT estimation is described in Dahan, Greenwald, Olofsen, Duma, “Pulse Transit Time (PTT) Reflects Changes in Anesthetic State During Sevoflurane/N₂O Anesthesia,” Anesthesiology 2002; 96: A544. A study of 42 patients undergoing general anesthesia using sevoflurane/N20 validated the efficacy of PTT to reflect changes in arousal state and perceived surgical stimulation compared to traditional measures including heart rate (HR) and Bispectral Index (BIS) as well as Heart Rate Variability (HRV). ECG and finger SaO2 plethysmograph waveforms were continuously monitored as illustrated in FIG. 5. The method of the present invention was used to calculate the PTT. The average and standard deviation of intra-beat intervals over the preceding 30 seconds were used to estimate heart rate and Heart Rate Variability, respectively.

PTT increased during anesthetic induction (#1) and decreased during recovery (#4) as illustrated in FIG. 5 which shows sample patient trends. PTT (mean (SD)) was shorter in light hypnotic levels as measured by BIS >70 (i.e., 281 (17) msec) than deeper hypnotic levels (i.e., BIS <70:306 (20)msec, p <0.001). Inspection of patient trends demonstrated that PTT rapidly decreased in response to painful stimulation (e.g., during intubation (#2) and patient movement (#3)). As shown in the Table 1 below, PTT correlated more strongly with an objective measure of consciousness (BIS) (R=−0.52) than did heart rate or heart rate variability. These results demonstrate that PTT reflects changes in arterial tone resulting from changes in consciousness level (i.e., BIS) and inadequacy of analgesia. Rapid decreases in PTT reflect acute arterial constriction and occur during instances of perceived painful stimulation or recovery from anesthesia.

TABLE 1
Correlation Between Various Metrics of Consciousness

| | BIS | PTT | HRV | HR |
|---|---|---|---|---|
| BIS | — | −0.52 | 0.26 | 0.19 |
| PTT | | | n.s. | −0.42 |
| HRV | | | | −0.42 |

Clinicians may interpret the instantaneous PTT value directly or in context of its recent trend. The PTT (measured from the R-wave to the point of steepest ascent in the finger PPG waveform) in awake, normal subjects is typically 250 msec. The goal of adequate analgesia is to titrate sufficient analgesics to ensure that PTT is maintained greater than 250 msec. If there is a rapid decrease in PTT much less than 250 msec when the patient should be unconscious and free of stress and pain, then supplemental analgesics are administered to bring PTT greater than or equal to 250 msec.

The forgoing clinical algorithm may be modified to provide patient-specific titration of analgesia by replacing the population normal value of 250 msec with a patient specific value calculated during awake baseline monitoring.

Since PWV is linearly related to PTT, this invention includes the monitoring of PWV as a means to quantify level of stress, pain and arousal.

While the foregoing invention has been described with reference to its preferred environments, various alterations and modifications will occur to those skilled in the art. All such alternatives and modifications are intended to fall within the scope of the appended claim.

We claim:

1. A method of noninvasively monitoring and controlling stress, pain or arousal states during sedation or anesthesia comprising the steps of:
acquiring at least one ECG signal from a subject being analyzed;
acquiring an arterial pulse waveform;
processing said at least one ECG signal to identify a pulse initiation fiducial point;
processing said arterial pulse waveform to identify a pulse arrival fidicual point;
calculating the time difference between said pulse initiation fiducial point and said resultant pulse arrival fiducial points of cardiac cycles;
estimating a current PTT from a sequence of said time differences; and
adjusting the administration of analgesia based on clinical interpretation of PTT.

2. The method of claim 1 wherein said arterial pulse waveform is acquired through use of a photoplethysmograph.

3. The method of claim 1 wherein said arterial pulse waveform is acquired through use of a tonometer device.

4. The method of claim 1 wherein said arterial pulse waveform is acquired through use of an invasive arterial line.

5. The method of claim 1 wherein said pulse initiation fiducial point is determined by use of QRS detection.

6. The method of claim 1 wherein said pulse arrival fiducial point is determined by use of Pulse detection.

7. The method of claim 1 wherein the step of calculating the time difference between said pulse initiation fiducial point and said resultant pulse arrival fiducial point of a cardiac cycle further comprises the steps of:
for a pulse initiation fiducial point, identifying a resultant pulse arrival fiducial point within the following predetermined time interval;
if a pairing is identified, calculating the time difference between said initiation fiducial point and said arrival fiducial point; and
if a pairing is not identified, excluding data related to said pulse initiation fiducial point from further processing.

8. The method of claim 1 wherein said step of estimating said current PTT from a sequence of said time differences further comprises using the most recent value.

9. The method of claim 1 wherein said step of estimating said current PTT from a sequence of said time differences further comprises using the X% trim-mean over the last Y seconds, where X is 50% or 75%, and Y is between 5 and 30 seconds.

10. The method of claim 1 wherein said step of estimating said current PTT from a sequence of said time differences further comprises using median filtering over the last Y seconds where Y is between 5 and 30 seconds.

11. The method of claim 1 wherein said step of adjusting the administration of analgesia via clinical interpretation of PTT further comprises the step of:
if PTT decreases to less than a baseline value in response to surgical or procedural stimulation, then administering sufficient analgesia to increase PTT to greater than said baseline value.

12. The method of claim 7 wherein said predetermined time interval is 500 msec.

13. A system for noninvasively monitoring stress, pain or arousal in a subject comprising:
at least one ECG lead connected to a subject for acquiring ECG signals from said subject;
probe connected to a subject for acquiring pulse waveform signal from said subject;
a processor for analyzing said ECG and PPG signals to compute an estimate of said subject's PTT from the heart of said subject to a location on the body of said subject where said PPG probe is attached and for determining whether the administration of analgesia needs to be adjusted based on said PTT.

14. The system for noninvasively monitoring stress, pain or arousal in a subject of claim 13 wherein said probe is a photoplethysmograph.

15. The system for noninvasively monitoring stress, pain or arousal in a subject of claim 13 wherein said probe is a tonometer device.

16. The system for noninvasively monitoring stress, pain or arousal in a subject of claim 13 wherein said probe is a an invasive arterial line.
